(19) United States
(12) Patent Application Publication
(10) Pub. No.: US 2016/0120694 A1
(43) Pub. Date: May 5, 2016

(54) INJECTION INSTRUMENT AND METHOD

(71) Applicants: Matthew Edward Carr, New Hope, MN (US); Maria Katherine Crandall, Hugo, MN (US); Alyssa Marie Hennen, Waconia, MN (US); Dara David Koozekanani, Minneapolis, MN (US); Benjamin Schlichtmann, Excelsior, MN (US); Emily Marie Weiss, Austin, MN (US)

(72) Inventors: Matthew Edward Carr, New Hope, MN (US); Maria Katherine Crandall, Hugo, MN (US); Alyssa Marie Hennen, Waconia, MN (US); Dara David Koozekanani, Minneapolis, MN (US); Benjamin Schlichtmann, Excelsior, MN (US); Emily Marie Weiss, Austin, MN (US)

(21) Appl. No.: 14/531,497

(22) Filed: Nov. 3, 2014

(51) Int. Cl. A61F 9/00

(52) U.S. Cl. CPC A61F 9/0008; A61F 9/0026; A61M 5/427

(57) ABSTRACT

An injection instrument that includes a saddle configured to receive a syringe aligned on a saddle axis; a linear member coupled to the saddle at a sliding joint, the linear member having a first end and a second end, the sliding joint allowing relative movement between the linear member and the saddle in a direction aligned with a slide axis, the slide axis parallel with the saddle axis; and a guide member located at the second end, the guide member including an edge spaced apart from the saddle axis by a prescribed distance, the guide member including a tissue contact surface, wherein the tissue contact surface is located on a single side of the saddle axis.

### BACKGROUND

Macular Degeneration is a common disease of the retina in people over the age of 60. It involves the growth of vasculature and occlusions near the macula (center of the retina) and can lead to complete blindness. It is a very significant and growing problem in the United States and worldwide.

Injections for wet age-related macular degeneration (wet-AMD) are typically made 3.5-4 mm outside of the limbus (the border between the cornea and the sclera). The variability between these measurements can be based on whether the patient has previously had their lens replaced due to cataracts. If the lens has not been replaced, the physician administering the wet AMD treatment injections generally uses the 3.5 mm distance. If the lens has been replaced, the physician must take caution not to damage the lens, and can use the 4 mm distance.

Current injection treatment procedures to help slow progression of the disease can sometimes be inaccurate, can cause too much contact with the eye, and can lead to severe consequences such as retinal detachments.

### OVERVIEW

The present inventors have recognized the need for a quick, accurate, safe, comfortable, and inexpensive injection instrument and method of use. An approach for providing an accurate, quick injection placement is described below. The injection instrument and method can be used by ophthalmologists, medical professionals, nurses, and technicians who administer injections for treating wet-AMD or other ocular conditions. The device and method can give consistent results and therefore can be useful for ophthalmologists of all experience levels. Medical schools and other training facilities can also find a use for this device, as it is designed to improve injection accuracy and efficiency, benefiting practitioners and patients alike. Additionally, the device can be used in other ocular injection procedures, such as retinal vein occlusion and diabetic retinopathy. The injection locations of these procedures are typically also 3.5 mm outward from the limbus, the only difference being the medication injected.

Although the injection instrument and method will be described as a tool to help physicians or others accurately place intravitreal ocular injections, the injection instrument and method can be used in other locations of the bodies of human or animal patients without changing the intent of this disclosure.

In an example, the injection instrument can include an assembly having a saddle and a linear member. The saddle can include a syringe sheath that can be cylindrical and can include a radius of about 9 mm that can securely snap onto a syringe barrel. The arrangement of the syringe and saddle provides a space at the tip of the needle for a user to view an injection site. The length of the saddle can be about 40 mm so that the saddle does not interfere with the observation of measurement markings on the syringe. The length of the saddle is also configured such that it does not hinder a user's view of the syringe needle or the guide member. The saddle can include a saddle axis that can be parallel to a center longitudinal axis of the syringe needle (needle axis). The saddle axis and the needle axis can be collinear.

In an example, the saddle can be slidably coupled with a linear member at a sliding joint. The linear member can extend from a first end to a second end, having a length that can be about 110 mm. The first end of the linear member can be unobstructed such that the saddle can be assembled onto the linear member. The saddle member can translate relative to the linear member along a slide axis. The slide axis can be generally parallel to the saddle axis. During the injection procedure, the injection instrument can be held normal to the eye or at an angle that can vary within a 60 degree cone having an apex at the needle puncture point and a centerline normal to the eye. The injection procedure can proceed after the needle penetrates the eyeball surface.

In an example, the sliding joint between the saddle and the linear member can be a mating connection such as having a linear member with a rectangular cross section and the saddle member defining a rectangular tubular cavity that can translate along a portion of the linear member smoothly without friction.

In an example, the linear member can include a guide member located near the second end. The guide member can include a tissue contact surface having an oval shape. The guide member can include a guide surface wall or an edge that can be spaced apart from the needle axis a prescribed distance. A user can place a drop of iodine on the eye. The guide surface can be placed on or near a guide reference, such as the limbus of an eye. The guide surface, being a prescribed distance from the needle axis, can accurately aid a user in the placement of the tip of a needle during an injection procedure. With the guide member located in the above manner, the saddle, holding a syringe, can be moved downwardly towards the eye. The syringe needle can puncture the eye, the plunger can be depressed, and an injection can be delivered to the eye. The entire injection instrument and syringe can be discarded after use.

During an assembly of an injection instrument, a cap can be secured to the first end of the linear member to retain the saddle on the linear member. The cap can include a recessed portion, a notch, or clearance, to ensure that a flange of a syringe plunger can remain unobstructed.

This overview is intended to provide an overview of subject matter of the present patent application. It is not intended to provide an exclusive or exhaustive explanation of the invention. The detailed description is included to provide further information about the present patent application.

To further illustrate the injection instrument and method disclosed herein, a non-limiting list of examples is provided here:

In Example 1, an injection instrument can comprise: a saddle configured to receive a syringe aligned on a saddle axis; a linear member coupled to the saddle at a sliding joint, the linear member having a first end and a second end, the sliding joint allowing relative movement between the linear member and the saddle in a direction aligned with a slide axis, the slide axis parallel with the saddle axis; and a guide member located at the second end, the guide member including an edge spaced apart from the saddle axis by a prescribed distance, the guide member including a tissue contact surface, wherein the tissue contact surface is located on a single side of the saddle axis.

In Example 2, the injection instrument of Example 1 can optionally be configured such that when viewed from the second end towards the first end, the tissue contact surface has a shape that is one of elliptical, crescent shaped, circular, and a regular polygon.

In Example 3, the injection instrument of Example 1 can optionally be configured such that when viewed from the second end towards the first end, the tissue contact surface has a shape that is one of an irregular polygon shape, an irregular shape having curved edges, and an irregular shape having a curved edge and a straight edge.

In Example 4, the injection instrument of any one or any combination of Examples 1-3 can optionally be configured such that the saddle includes a syringe sheath conforming to an outer surface of a barrel of the syringe, the syringe sheath configured to secure the syringe with a snap fit.

In Example 5, the injection instrument of any one or any combination of Examples 1-4 can optionally be configured such that the edge is separated from the slide axis by an offset.

In Example 6, the injection instrument of any one or any combination of Examples 1-5 can optionally be configured such that the tissue contact surface is located wholly within a 120 degree sector of a circular area, the circular area having a center located at the saddle axis and the circular area in a plane perpendicular to the saddle axis.

In Example 7, the injection instrument of any one or any combination of Examples 1-6 can optionally be configured such that the saddle extends from a plunger end to a needle end with a length in a range of about 30 mm to about 50 mm.

In Example 8, the injection instrument of any one or any combination of Examples 1-7 can optionally be configured such that the edge is formed by a junction of the tissue contact surface and a guide surface wall.

In Example 9, the injection instrument of any one or any combination of Examples 1-8 can optionally be configured such that the saddle and the linear member are one of polyethylene, polyvinyl chloride, polypropylene, or polystyrene.

In Example 10, the injection instrument of any one or any combination of Examples 1-8 can optionally be configured such that the prescribed distance is in a range from about 3 mm to about 5 mm.

In Example 11, the injection instrument of any one or any combination of Examples 1-10 can optionally be configured such that the prescribed distance is measured from saddle axis to an outermost point of the tissue contact surface.

In Example 12, the injection instrument of Example 11 can optionally be configured such that the outermost point of the tissue contact surface is a point on the tissue contact surface closest to the slide axis and farthest from the saddle axis.

In Example 13, the injection instrument of any one or any combination of Examples 1-12 can optionally be configured such that the edge includes an outermost point located the prescribed distance from the saddle axis.

In Example 14, the injection instrument of any one or any combination of Examples 1-13 can optionally be configured such that the sliding joint includes a keyed connection configured to preclude rotation of the saddle relative to the linear member.

In Example 15 an injection instrument can comprise: a saddle member extending in a longitudinal direction from a first end to a second end, the saddle configured to receive a syringe barrel aligned on a saddle axis, the saddle configured to slidably engage the syringe barrel; a support member fixedly coupled to the saddle, the support member extending in the longitudinal direction from a support member first end to a support member second end; and a guide member located at the support member second end, the guide member having an edge that is spaced apart from the saddle axis a prescribed distance, the guide member including a tissue contact surface, wherein the tissue contact surface is located on a single side of the saddle axis.

In Example 16 a method of providing an injection can comprise: securing a barrel of a syringe in a saddle of an injection device, the saddle including a saddle axis that is coaxial with a syringe needle axis, the saddle slidably coupled to a linear member; while aligning an edge of a guide member with a guide reference on a surface, placing the guide member against the surface, wherein the edge is spaced apart from the saddle axis a prescribed distance; sliding the saddle along a slide axis of the linear member, the linear member connected to the guide member, wherein the slide axis is parallel to the saddle axis; puncturing the surface with a syringe needle connected to the barrel; and pushing a syringe plunger towards the surface to deliver the injection.

In Example 17, the method of Example 16 can optionally be configured such that placing the guide member against the surface does not include touching a corneal surface.

In Example 18, the method of any one or any combination of Examples 16-17 can optionally be configured to comprise holding the injection device such that the syringe needle axis falls within a 60 degree cone normal to the surface having an apex at a needle puncture point.

In Example 19, the method of any one or any combination of Examples 16-18 can optionally be configured such that the guide member is located wholly within a 120 degree sector of a circular area, the circular area having a center located at the saddle axis and the circular area in a plane perpendicular to the saddle axis.

In Example 20, the method of any one or any combination of Examples 16-19 can optionally be configured such that the prescribed distance is in a range from about 2 mm to about 5 mm.

In Example 21, the injection instrument or method of any one or any combination of Examples 1-20 can optionally be configured such that all elements, operations, or other options recited are available to use or select from.

These and other examples and features of the present injection instrument and method will be set forth in part in the following Detailed Description. This Overview is intended to provide non-limiting examples of the present subject matter—it is not intended to provide an exclusive or exhaustive explanation. The Detailed Description below is included to provide further information about the present injection instrument and method.

### BRIEF DESCRIPTION OF THE DRAWINGS

FIG. 1 illustrates a schematic view of an eyeball in accordance with at least one example of the present subject matter.

FIG. 2 illustrates a perspective view of an injection instrument in accordance with at least one example of the present subject matter.

FIG. 3 illustrates a perspective view of a linear member in accordance with at least one example of the present subject matter.

FIG. 4 illustrates a bottom view of the linear member in accordance with at least one example of the present subject matter.

FIG. 5 illustrates a perspective view of a saddle in accordance with at least one example of the present subject matter.

FIG. 6 illustrates a cross sectional view of the injection instrument illustrated in FIG. 2 in accordance with at least one example of the present subject matter.

FIG. 7 illustrates a perspective view of a cap in accordance with at least one example of the present subject matter.

FIG. 8 illustrates a schematic view of an injection site in accordance with at least one example of the present subject matter.

FIG. 9 illustrates a side view of a one piece injection instrument in accordance with at least one example of the present subject matter.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

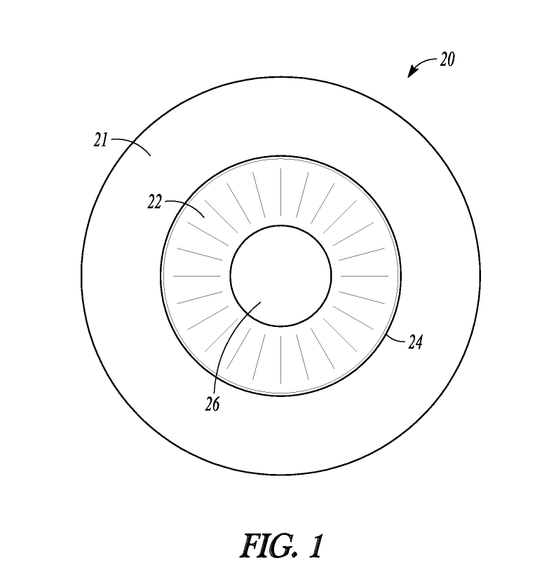

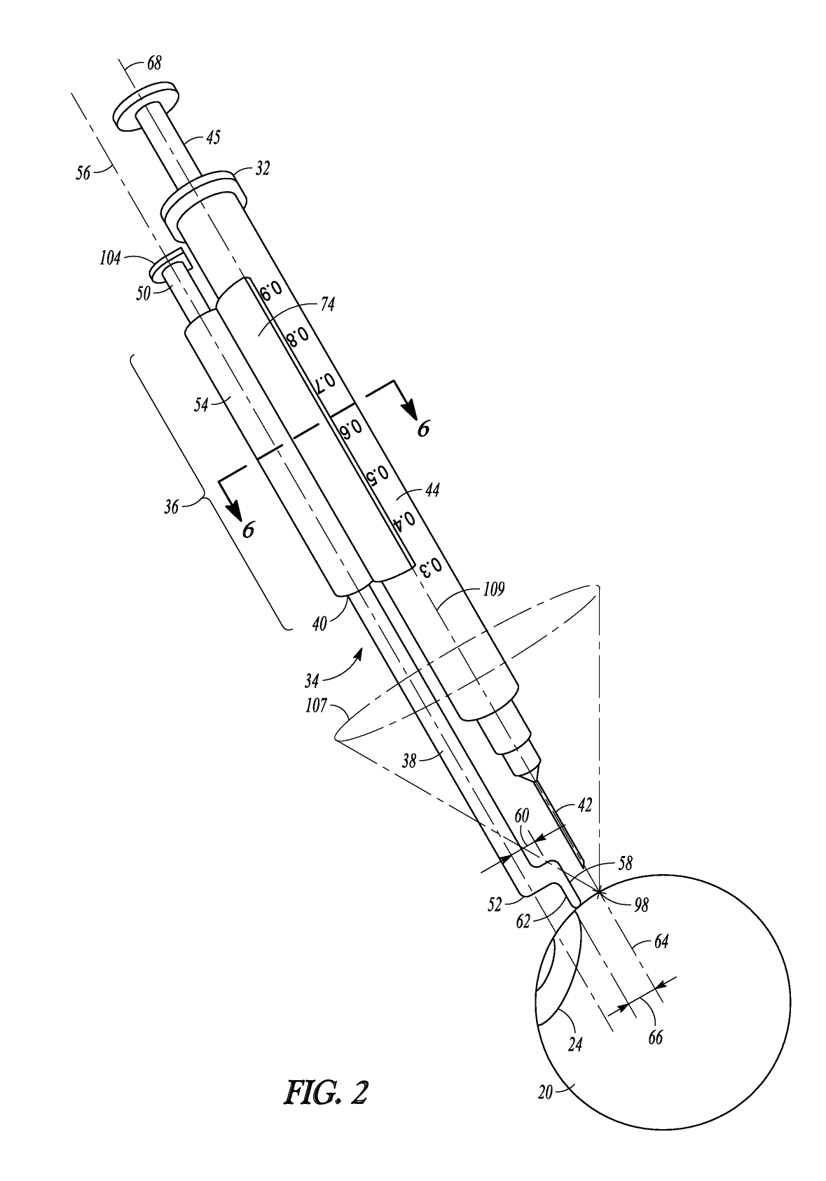

FIG. 1 illustrates a schematic view of an eye 20. The eye 20 can include sclera region 21 (also known as the white of the eye 20). The sclera region 21 can surround an iris 22 which is commonly known as the colored portion of the eye 20. A boundary, known as the limbus 24 can be formed at the junction between a transparent cornea 26 and the sclera region 21. The transparent cornea 26 can be located inwardly from the limbus 24. The limbus 24 can also mark the outer edge of the iris 22. The limbus 24 can be used as a guide reference for placement of an injection. Although a human eye is shown, an animal eye is also contemplated by the inventors without changing the intent of this disclosure. Although in this example, the guide reference is the limbus 24 of the eye 20, other locations of a human or animal body can be used as a guide reference to accurately position the location of an injection instrument 34 (see FIG. 2). Although some examples of this disclosure describe an injection to the eye 20, an injection instrument 34 described herein can also be used for other parts of the anatomy without changing the intent of this disclo sure.

FIG. 2 illustrates a perspective view of an injection instrument 34 in accordance with at least one example of present subject matter. FIG. 2 illustrates a schematic view of an eye 20 and the injection instrument 34 to show an example of usage. The proportions, angles, and viewpoints illustrated are for descriptive purposes only and should not be construed as limiting the scope of this disclosure. The injection instrument 34 can be used in conjunction with a syringe 32. The injection instrument 34 can include a saddle 36 and a linear member 38 that can be connected by a sliding joint 40.

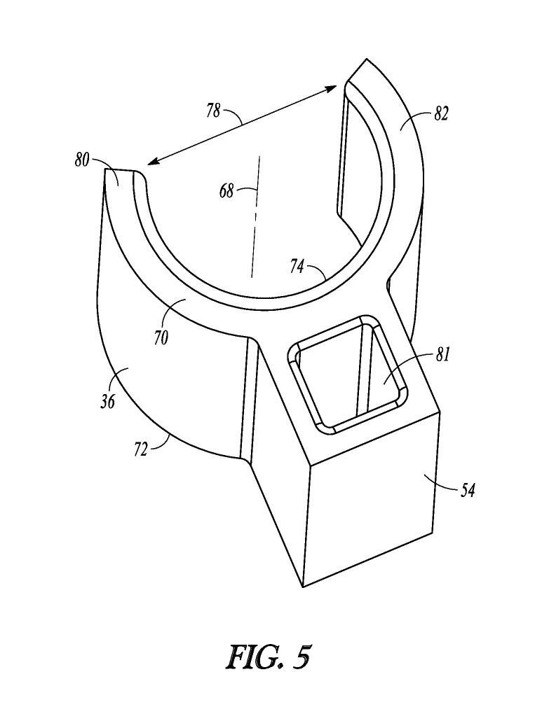

The syringe 32 can include a needle 42, a syringe barrel 44 and a plunger 45. The syringe barrel 44 can be received by the saddle 36. The saddle 36 can define a syringe sheath 74 having a semi-cylindrical shape (see FIG. 5). In a saddle 36 having a semi-cylindrical shaped syringe sheath 74, a saddle axis 68 can be centered in the syringe sheath 78. When a syringe 32 has been fit into the saddle 36, a needle axis and the saddle axis 68 can be coaxial. In an example, the saddle 36 can be configured so that when a syringe barrel 44 is pressed against the saddle 36, the syringe barrel 44 can be received into the saddle 36 in a manner such as a snap fit that securely holds the syringe barrel 44 to the saddle 36. In other configurations, the syringe 32 can be attached to the saddle 36 such as by a strap, a band, or a fastener. The fastener can be a screw, bolt, push pin, or clip. In an example, the syringe barrel 44 can be integrally formed into a saddle shaped configuration.

The saddle 36 can include a connection portion 54, which can extend outwardly from the syringe sheath 74. The connection portion 54 can be coupled with a linear member 38 by the sliding joint 40. The linear member 38 can extend from a first end 50 to a second end 52, and can provide a rail or track, upon which the saddle 36 can slide. A cap 104 can be secured onto the first end 50, by glue, adhesive, welding, a fastener, or other attachment means. The cap 104 can prevent the saddle 36 from disassembling from the linear member 38 during transport, packaging or use.

In an example, the connection portion 54 can be located on a side of the saddle 36 that is opposite a side that engages the syringe barrel 44. In an example, the connection portion 54 can be located in other orientations to the syringe barrel 44, such as to one or more sides of the syringe barrel 44. Although the injection instrument 34 is illustrated as including one linear member 38, in another example, a plurality of linear members 38 can be coupled with the saddle 36 by a sliding joint 40. The sliding joint 40 can allow the saddle 36 to translate along a slide axis 56 that can be parallel to or coincide with a center longitudinal axis of the linear member 38. The saddle 36 can translate from the first end 50 to the second end 52. In an example, the sliding joint 40 can be smooth in operation with little friction between the moving parts. In another example, the fit between the linear member 38 and the saddle 36 can be calibrated to provide an amount of drag between the moving parts. The drag can be accomplished by tightness of fit between the saddle 36 and linear member 38 or raised structures such as ridges or protrusions on one or both of the saddle 36 or linear member 38.

The linear member 38 can include a guide member 58 located near the second end 52. Portions of the guide member 58 can be separated laterally from the slide axis 56 by an offset 60, to provide a location of the guide member 58 that can aid in location of a needle puncture point 98. The needle puncture point 98 can be a location that the needle 42 can puncture the eye 20. In the example of an injection for wet age-related macular degeneration, the needle puncture point 98 is typically made 3.5 mm to 4 mm from the limbus 24. Portions of the guide member 58 can be used as a visual and structural guide for placement of the injection instrument 34 on the eye 20 and delivery of an injection.

Referring to FIGS. 2, 3, and 4, a portion of the guide member 58 can be configured as an elliptical cylinder having an oval shaped tissue contact surface 100 at a distal end 61 configured to touch the eye 20. A guide surface wall 62 of the guide member 58 can intersect the tissue contact surface 100 at an edge 102. The shape of the guide member 58 and the edge 102 can be used as a visual and structural guide for placement of a portion of the edge 102 against or very near to the limbus 24. Such a placement can ensure that the injection instrument 34 can locate a needle puncture point 98 at a suitable location on the eye 20. The location of the needle puncture point 98 can be dependent on a prescribed distance 66 that separates a point or area on the edge 102 and the needle axis 64. The edge 102 is illustrated as having a convex curve. An outermost point 101 of the edge 102 in a direction away from the needle axis 64 (see also FIG. 8), can be used to align with the limbus 24.

Other shapes of the guide member 58 and the tissue contact surface 100 are contemplated without changing the intent of the disclosure. In this disclosure an irregular shape, can include an irregular polygon, a shape having both curved and straight portions, a shape having both convex and concave curves, or a shape having an edge without repeating patterns. In an example, the guide member 58 can include a cross section that can be elliptical, crescent shaped, circular, a regular polygon, an irregular polygon shape, an irregular shape having curved edges, an irregular shape having a curved edge and a straight edge, or combinations of the aforementioned shapes. In an example, the tissue contact surface 100 can include shapes that are elliptical, crescent shaped, circular, a regular polygon, an irregular polygon shape, an irregular shape having curved edges, an irregular shape having a curved edge and a straight edge or combinations of the aforementioned shapes. The tissue contact surface 100 can include an edge 102 that can be shaped as a concave curve to generally match a radius of the limbus 24. In an example, the edge 102 can be straight or have a pointed shape.

A portion of the guide surface wall 62 and the edge 102, such as the outer most point 101 can be spaced apart from a needle axis 64 the prescribed distance 66. The prescribed distance 66 can vary according to what type of procedure the user intends to accomplish and configurations of the injection instrument 34 can be formed accordingly. The prescribed distance 66 can ensure that the needle puncture point 98 can be located at the prescribed distance 66 from the limbus 24. The needle axis 64 can be parallel to the slide axis 56. In an example, in a configuration in which the saddle 36 can be non-cylindrical, the needle axis 64 and the saddle axis 68 can be parallel and non-coaxial.

To perform an injection procedure, the user can grasp the injection instrument 34 and/or the syringe 32 and can place the edge 102 of the guide wall surface 62 near, adjacent, or on a guide reference such as the limbus 24. In an example, the outermost point 101 can be aligned with the limbus 24. The guide member 58 need not contact the cornea 26. Such a lack of contact can avoid corneal abrasion. During the injection procedure, the injection instrument 34 can be held such that the needle axis 64 is normal 109 to the eye 20. In an example, the injection instrument 34 can be held at an angle such that the needle axis 64 falls within a 60 degree cone 107 having an apex at the needle puncture point 98 and a centerline normal 109 to the eye 20. The saddle 36 can be translated towards the eye 20, or other such surface needing an injection, to allow the tip of the needle 42 to puncture the eye 20. The plunger 45 can be depressed and an injection solution contained by the syringe 32 can be delivered to the eye 20. The saddle 36 can be configured with an opening 78 (see FIG. 5) so that markings on the syringe barrel 44 can be visible so the user can administer a particular injection dosage. The injection instrument 34 can be formed of plastic, polymer, metal, wood, composites, rubber, or combinations of the aforementioned materials. The injection instrument 34 can be formed of polyethylene, polyvinyl chloride, polypropylene, or polystyrene. The injection instrument 34 can be formed by injection molding.

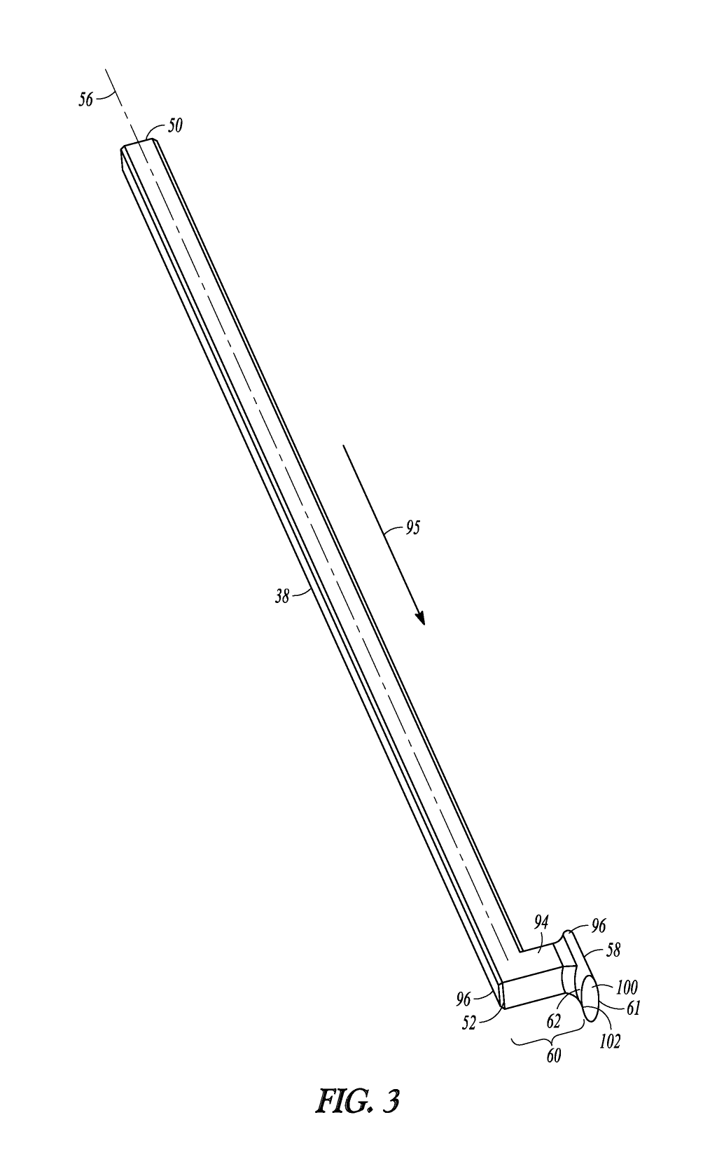

FIG. 3 illustrates a perspective view of a linear member 38 in accordance with at least one example of present subject matter. The linear member 38 can include a horizontal member 94 that can connect the guide member 58 to the second end 52. The guide member 58 can extend farther distally 95 than the second end 52, to the distal end 61. The horizontal member 94 can provide the offset 60 that can locate the guide surface wall 62 away from the slide axis 56, the prescribed distance 66 from the needle axis 64. Dimensions and configurations of the offset 60 can be dependent on the dimensions of the syringe 32 and the prescribed distance 66 needed for a particular application. Although the connections between the second end 52 and the horizontal member 94 and the horizontal member 94 and the guide member 58 are illustrated as having right angles 96, the connections can be made in any form so as to laterally displace the guide surface wall 62 from the slide axis 56. The linear member 38 can include a horizontal member 94 and/or guide member 58 that are integrally formed and can include curved shapes rather than right angles 96. The guide member 58 and the horizontal member 94 can be unitary and the angle of the horizontal member 94 need not be 90 degrees from the slide axis 56. The guide member 58 can include the tissue contact surface 100 that can be facing in a direction opposite the first end 50. The tissue contact surface 100 can be placed against the anatomy of a human or animal body such as against the surface of the eye 20. As such, the guide member 58 can be configured in a wide array of shapes to maintain the prescribed distance 66 between the guide surface wall 62 and the needle axis 64.

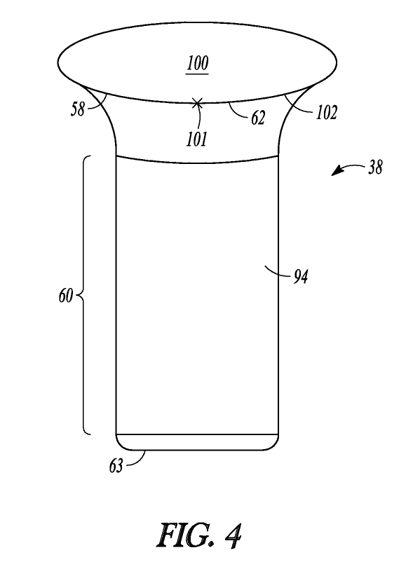

FIG. 4 illustrates a bottom view of the linear member 38 in accordance with at least one example of present subject matter. FIG. 4 illustrates a view of the linear member 38 of FIG. 3 as viewed from the second end 52 towards the first end 50. A junction of the tissue contact surface 100 and the guide surface wall 62 can form an edge 102. The edge 102 can include a smooth rounded radius that can prevent any damage to the surface of the eye 20. The guide surface wall 62 and the edge 102 can be used as a visual or structural guide to place the tissue contact surface 100 on, adjacent, or near a guide reference such as the limbus 24. Although the guide surface wall 62 is illustrated as the surface of the guide member 58 that is closest to a linear member end 63 of the offset 60, in an example, the guide member 58 can be configured in any orientation that will allow a user to use a portion of its geometry as a guide to placement of the injection instrument 34. The horizontal member 94 is illustrated as rectangular, however, in an example, the horizontal member 94 can be curvilinear and shaped in any manner to position the guide member 58 in position relative to the needle axis 64. The linear member 38 and the slide axis 56 can be adjacent and parallel to the needle axis 64 (see FIG. 8). Various configurations of the guide member 58 in relation to the linear member 38, such as straight or curved, longer or shorter, can allow the slide axis 56 to be positioned anywhere within 360 degrees of the needle axis 64. Such various configurations can still allow the prescribed distance 66 to be maintained.

FIG. 5 illustrates a perspective view of a saddle 36 in accordance with at least one example of present subject matter. The saddle 36 can extend from a plunger end 70 to a needle end 72. The saddle 36 can define an opening 78 that can extend from the plunger end 70 to the needle end 72. The opening 78 can be bordered by a first flange 80 and a second flange 82. The opening 78 can be sized and shaped such that the syringe barrel 44 can be pressed into the opening 78 and be snap fit into the syringe sheath 74. The first flange 80 and the second flange 82 can be formed from a material that can flex outwardly when a largest diameter of the syringe barrel 44 passes through the opening 78 and then the flanges 80, 82 can return to an original shape and securely hold the syringe barrel 44 to the saddle 36. In another example, the saddle 36 can have a receiving shape that is not cylindrical, such as “U”, “L”, or “V” shaped in cross section and the syringe 32 can be received into a trough of these shapes. In a saddle 36 having an alternative shape, such as the “U”, “V”, or “L” shape, the saddle axis 68 can be parallel to the slide axis 56, but may not be centered. The saddle 36 can be formed of a shape memory material that can flex and return to its original shape.

The connection portion 54 can be located opposite from the syringe sheath 74 and can extend from the plunger end 70 to the needle end 72. The connection portion 54 can define a passageway 81 that can receive the linear member 38. The passageway 81 can define a rectangular opening as shown in the figure and can couple with the linear member 38 by the sliding joint 40. Although the saddle 36 is illustrated as unitary, it can be formed of a plurality of parts.

The length of the saddle 36 from the plunger end 70 to the needle end 72 can be about 40 mm so that the saddle 36 does not interfere with the observation of measurement markings on the syringe 32. The length of the saddle is also configured such that it does not hinder a user's view of the needle 42 or the guide member 58. The length of the saddle can range from about 30 mm to about 50 mm.

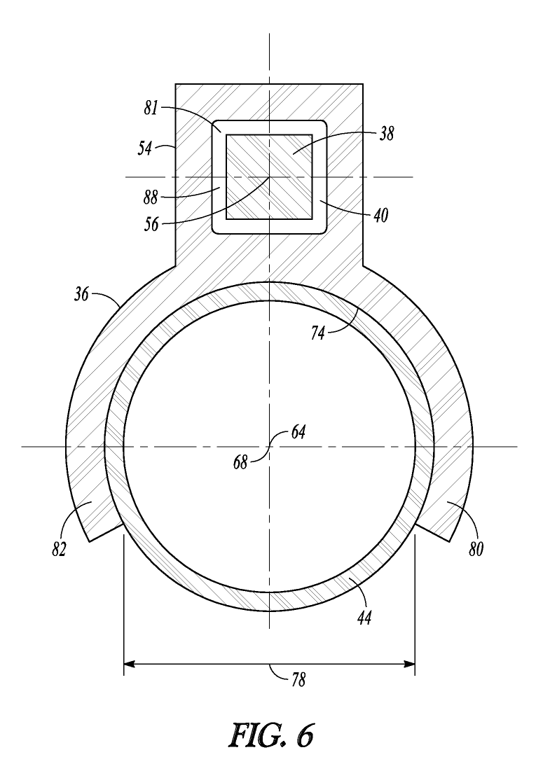

FIG. 6 illustrates a cross sectional view of the injection instrument 34 illustrated in FIG. 2. in accordance with at least one example of the present subject matter. The sliding joint 40 is shown as having a rectangular shaped linear member 38 fitting within the passageway 81 of the connection portion 54 of the saddle 36. Although the sliding joint 40 is illustrated as having a rectangular linear member 38 sliding within the passageway 81 of the saddle 36, the sliding joint 40 can be configured with other complementary mating shapes, such as elliptical, cross-shaped, crescent shaped, L-shaped, V-shaped, irregularly shaped, or combinations of shapes. Any of the foregoing complementary mating shapes can form a keyed connection 88 which can prevent the saddle 36 from rotating about the slide axis 56 of the linear member 38. The mating shapes could also be reversed, such as by having a connection portion 54 that is solid and rectangular, sliding within a passageway configured into a linear member 38.

The opening 78, first flange 80 and second flange 82 can form the syringe sheath 74 that can receive the syringe barrel 44. Although the connection portion 54 is illustrated as centered between the first flange 80 and second flange 82, the connection portion 54 can be located in any orientation of the saddle 36 to provide a sliding joint 40 with the linear member 38. FIG. 6 illustrates the saddle axis 68 coincident with the needle axis 64, and the slide axis 56 which can be perpendicular to the plane of the page of the illustration.

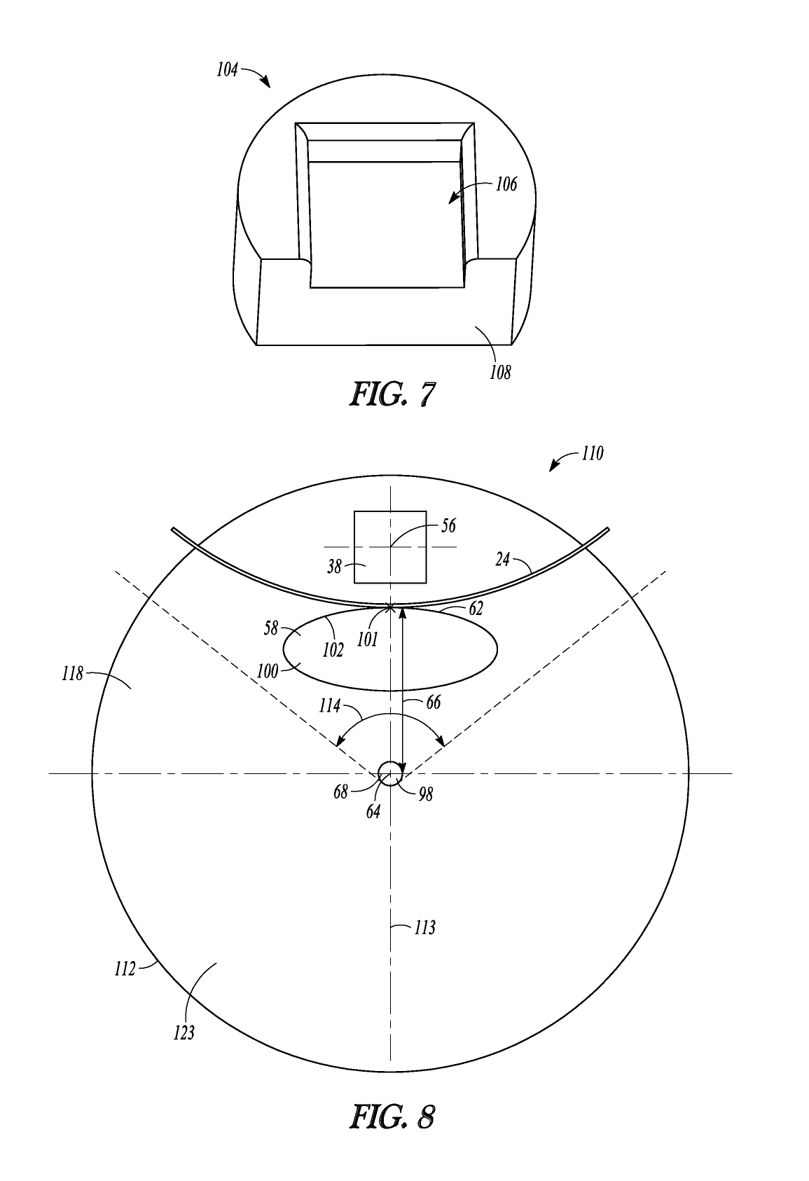

FIG. 7 illustrates a perspective view of the cap 104 in accordance with at least one example of present subject matter. The cap 104 can include a cap cavity 106 that can conform to the first end 50. The cap 104 can include a recessed side 108 that can provide a recessed portion, a notch, or clearance, to ensure that a flange of the plunger 45 can remain unobstructed.

FIG. 8 illustrates a schematic view of an injection site 110 in accordance with at least one example of present subject matter. A circular area 112 is illustrated for purposes of describing possible positions of the slide axis 56, the linear member 38, the guide member 58, and the tissue contact surface 100 (viewed from the top) in relation to the needle axis 64 and the diameter of the circular area 112 can be any dimension. The circular area 112 is illustrated having at its center, the needle axis 64. The circular area 112 is illustrated in a plane 117 perpendicular to the saddle axis 68. In an example, the linear member 38 with a slide axis 56 can be generally located in a line 113 including the guide member 58 and the needle puncture point 98. In an example, as mentioned above, the saddle axis 68 and the needle axis 64 can be coaxial. In an example, the guide member 58 can be positioned in relation to the saddle axis 68 such that the guide member 58 and/or the tissue contact surface 100 can be located wholly within a 120 degree sector 114 of the circular area 112.

In an example, the linear member 38 can be positioned such that a perpendicular intersection of the slide axis 56 can be located wholly within a first 180 degree sector 118 of the circular area 112, the first 180 degree sector 118 wholly including the guide member 58. In another example, the linear member 38 can be positioned such that a perpendicular intersection of the slide axis 56 can be located wholly within a second 180 degree sector 123 of the circular area 112, the second 180 degree sector 123 not including any portion of the tissue contact surface 100. These examples are given for illustration only and the slide axis 56 and linear member 38 can be positioned in any location within 360 degrees relative to the saddle axis 68, while still maintaining the slide axis 56 parallel to the saddle axis 68 and having the edge 102 connected to the linear member 38, such that the edge 102 is located a prescribed distance 66 from the saddle axis 68.

The edge 102 is illustrated as elliptical, but can be straight, concave, or irregularly shaped and can be shaped to match the shape of a guide reference, such as the limbus 24. The edge 102 can be used as a visual aid for a user to align the guide member 58 with a guide reference such as the limbus 24. In use, a portion of the edge 102, for example, the outermost point 101 can be placed on the eye 20 just adjacent to the limbus 24. In this manner, the tissue contact surface 100 need not contact the cornea 26 of the eye. Such a placement can locate the needle axis 64 the prescribed distance 66 from the limbus 24. The prescribed distance 66 can be measured from the saddle axis 56 to the outermost point 101. The outermost point 101 can be a point on the tissue contact surface 100 closest to the slide axis 56 and farthest from the saddle axis 68. Depending on the configuration of the injection instrument 34, a user can view the edge 102 from different angles to achieve placement of the injection instrument 34.

In an example, the prescribed distance 66 can be configured in a range of about 1 mm to about 1000 mm. In an example, the prescribed distance 66 can be configured in a range of about 1 mm to about 100 mm. In an example, the prescribed distance 66 can be configured in a range of about 1 mm to about 10 mm. In an example, the prescribed distance 66 can be configured in a range of about 1 mm to about 5 mm. In an example, the prescribed distance 66 can be configured in a range of about 2 mm to about 5 mm. In an example, the prescribed distance 66 can be configured in a range of about 3 mm to about 5 mm. In an example, the prescribed distance 66 can be configured in a range of about 3 mm to about 4 mm. In an example, the prescribed distance 66 can be configured to about 3.5 mm. In an example, the prescribed distance 66 can be configured to about 4 mm.

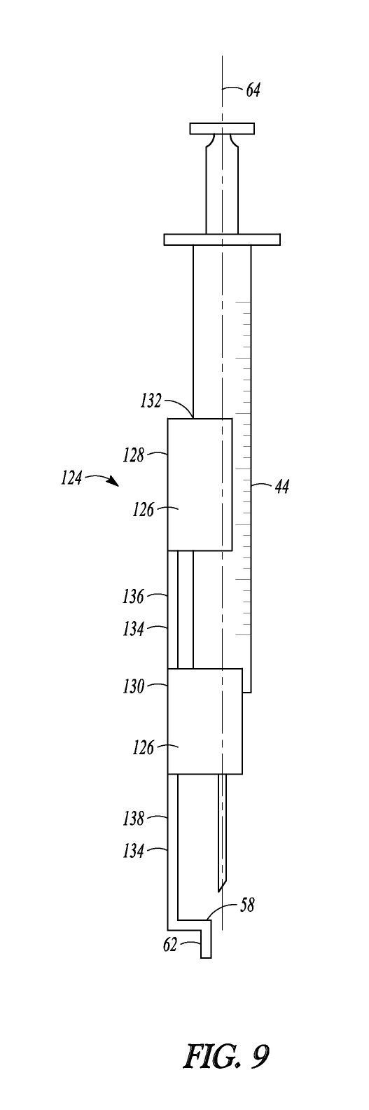

FIG. 9 illustrates a side view of a one piece injection instrument 124 in accordance with at least one example of present subject matter. The injection instrument 34 can be configured as a one piece injection instrument 124. In an example, the one piece injection instrument 124 can include one or more barrel slide members 126 and can include an upper barrel slide member 128 and a lower barrel slide member 130. The barrel slide members 126 can engage the syringe barrel 44 in a barrel sliding joint 132 whereby the syringe barrel 44 can translate upwardly or downwardly relative to the barrel slide members 126. The one piece injection instrument 124 can include one or more vertical support members 134 which can include a lower support member 138 and an upper support member 136. The one or more support member 134 can be fixedly connected to the barrel slide members 126. The lower support member 138 can include a guide member 58 that can provide the features mentioned above. An upper support member 136 can connect the upper barrel slide member 128 and the lower barrel slide member 130. In another example, the barrel slide members 126 can be a single piece and the upper support member 136 can be omitted. The one piece injection instrument 124 can include a needle axis 64 that can be spaced apart from a guide surface wall 62 a prescribed distance 66, such as described above. In operation, the guide surface wall 62 can be placed on or near a guide reference such as a limbus 24 and the syringe barrel 44 can be guided by the barrel slide members 126 to puncture a target location and the plunger 45 can deliver an injection.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An injection instrument comprising:
a saddle configured to receive a syringe aligned on a saddle axis;
a linear member coupled to the saddle at a sliding joint, the linear member having a first end and a second end, the sliding joint allowing relative movement between the linear member and the saddle in a direction aligned with a slide axis, the slide axis parallel with the saddle axis; and
a guide member located at the second end, the guide member including an edge spaced apart from the saddle axis by a prescribed distance, the guide member including a tissue contact surface, wherein the tissue contact surface is located on a single side of the saddle axis.

2. The injection instrument of claim 1, wherein when viewed from the second end towards the first end, the tissue contact surface has a shape that is one of elliptical, crescent shaped, circular, and a regular polygon.

3. The injection instrument of claim 1, wherein when viewed from the second end towards the first end, the tissue contact surface has a shape that is one of an irregular polygon shape, an irregular shape having curved edges, and an irregular shape having a curved edge and a straight edge.

4. The injection instrument of claim 1, wherein the saddle includes a syringe sheath conforming to an outer surface of a barrel of the syringe, the syringe sheath configured to secure the syringe with a snap fit.

5. The injection instrument of claim 1, wherein the edge is separated from the slide axis by an offset.

6. The injection instrument of claim 1, wherein the tissue contact surface is located wholly within a 120 degree sector of a circular area, the circular area having a center located at the saddle axis and the circular area in a plane perpendicular to the saddle axis.

7. The injection instrument of claim 1, wherein the saddle extends from a plunger end to a needle end with a length in a range of about 30 mm to about 50 mm.

8. The injection instrument of claim 1, wherein the edge is formed by a junction of the tissue contact surface and a guide surface wall.

9. The injection instrument of claim 1, wherein the saddle and the linear member are one of polyethylene, polyvinyl chloride, polypropylene, or polystyrene.

10. The injection instrument of claim 1, wherein the prescribed distance is in a range from about 3 mm to about 5 mm.

11. The injection instrument of claim 1, wherein the prescribed distance is measured from saddle axis to an outermost point of the tissue contact surface.

12. The injection instrument of claim 11, wherein the outermost point of the tissue contact surface is a point on the tissue contact surface closest to the slide axis and farthest from the saddle axis.

13. The injection instrument of claim 1, wherein the edge includes an outermost point located the prescribed distance from the saddle axis.

14. The injection instrument of claim 1, wherein the sliding joint includes a keyed connection configured to preclude rotation of the saddle relative to the linear member.

15. An injection instrument comprising:
a saddle member extending in a longitudinal direction from a first end to a second end, the saddle configured to receive a syringe barrel aligned on a saddle axis, the saddle configured to slidably engage the syringe barrel;
a support member fixedly coupled to the saddle, the support member extending in the longitudinal direction from a support member first end to a support member second end; and
a guide member located at the support member second end, the guide member having an edge that is spaced apart from the saddle axis a prescribed distance, the guide member including a tissue contact surface, wherein the tissue contact surface is located on a single side of the saddle axis.

16. A method of providing an injection comprising:
securing a barrel of a syringe in a saddle of an injection device, the saddle including a saddle axis that is coaxial with a syringe needle axis, the saddle slidably coupled to a linear member;
while aligning an edge of a guide member with a guide reference on a surface, placing the guide member against the surface, wherein the edge is spaced apart from the saddle axis a prescribed distance;
sliding the saddle along a slide axis of the linear member, the linear member connected to the guide member, wherein the slide axis is parallel to the saddle axis;
puncturing the surface with a syringe needle connected to the barrel; and
pushing a syringe plunger towards the surface to deliver the injection.

17. The method of claim 16, wherein placing the guide member against the surface does not include touching a corneal surface.

18. The method of claim 16, comprising holding the injection device such that the syringe needle axis falls within a 60 degree cone normal to the surface having an apex at a needle puncture point.

19. The method of claim 16, wherein the guide member is located wholly within a 120 degree sector of a circular area, the circular area having a center located at the saddle axis and the circular area in a plane perpendicular to the saddle axis.

20. The method of claim 16, wherein the prescribed distance is in a range from about 2 mm to about 5 mm.
